(19) United States
(12) Patent Application Publication
(10) Pub. No.: US 2004/0063915 A1
(43) Pub. Date: Apr. 1, 2004

(54) Metalization of microtubules

(76) Inventors: Bruce A. Diner, Chadds Ford, PA (US); Mark Andrew Harmer, Kennett Square, PA (US); Greg Mitchell, Wilmington, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/643,605

(22) Filed: Aug. 18, 2003

Related U.S. Application Data
(60) Provisional application No. 60/404,887, filed on Aug. 21, 2002.

(51) Int. Cl. C07K 16/46; C07K 14/195

(52) U.S. Cl. 530/391.1; 530/350; 530/356; 530/400

(57) ABSTRACT

Protein polymers coated with a metal are provided. New methods are described for the preparation of tubulin-based microtubules such that they may be coated, by an electroless plating method, with gold particles of <20 nm in diameter without prior reaction with noble metal catalysts. The gold particle deposition can occur in suspension or on microtubules lying on a surface. Gold enhancement of the gold-particle coating and annealing results in ohmic conductance that is within a factor of ten of the resistivity of bulk gold. These methods are likely to be applicable to other protein polymers.

This application claims the benefit of United States Provisional Application 60/404,887, filed Aug. 21, 2002.

### FIELD OF INVENTION

The invention relates to the field of nanotechnology. More specifically, linear, high aspect ratio protein polymers have been coated with a metal for use as nano-wires.

### BACKGROUND

The steady effort to increase the density of integrated circuits is being challenged by the limits of photolithographic techniques as the dimensions of electronic devices and interconnects reach, and attempt to fall below the wavelength of visible and UV light (The International Technology Roadmap for Semiconductors, 1999). The application of extreme UV, X-ray and electron lithographic techniques while likely to improve resolution, is expected to do so at considerable cost. New materials have been proposed to complement or even replace traditional silicon-based technology. Conducting organic materials and nanowires made of carbon nanotubes or of non-silicon semiconducting materials have attracted a great deal of attention among these alternatives. One of the great challenges to finding alternatives to photolithography is the need to be able to place devices and interconnects with precision to make a functional circuit. One approach is to harness biological molecules in this role, in attempting to exploit the self-assembling, self-directing techniques that nature uses to position critical molecules within the cell. Molecules such as DNA and the cytoskeletal proteins are particularly well-adapted to this application as their monomeric units can polymerize to form structures of high aspect ratio. They are also capable of being recognized with high specificity by other molecules. Both characteristics are desirable attributes for molecules to be fashioned into nanoscale interconnects.

Microtubules are composed of α/β-tubulin heterodimers that polymerize in the presence of guanosine triphosphate (GTP). The tubules are typically composed of 13-15 protofilaments, linear polymers of the α/β heterodimers arranged in a head to tail fashion (for review see Desai, A. and Mitchison, T (1997) Annu. Rev. Cell Dev. Biol. 13, 83-117). The protofilaments associate side to side α- to α- and β- to β- to form tubes that are 25 nm external diameter and 14 nm internal diameter and up to tens of microns in length. Tubulin (equimolar in α- and β-) is readily isolated and purified from bovine brain in large quantities (gram scale). Polymerization is rapid in vitro, with the composition of the suspension medium, the tubulin concentration and the incubation time controlling polymer length.

With the exception of biological molecules that contain redox active centers, most biological molecules are refractory to electron transfer. One solution to this deficiency is to develop methods for metalizing biological polymers, capable of forming 2- and 3-dimensional structures that can potentially form or link nanoelectronic devices. These polymeric molecules would, either naturally or following derivativization, have affinity for metal ions that, upon reduction, form a conductive metal cladding. Among these biological molecules that have been metalized in this way are lipid micelles (Schnur, J. M. et al. (1987) Thin Solid Films 152, 181-206; Markowitz, M. et al. (1992) Thin Solid Films 224, 242-7), rhapidosomes (Pazirandeh, M. et al. (1992) Biomimetics 1, 41), DNA (Braun, E. et al. (1998) Nature 391, 775-8) and microtubules (Kirsch, R. et al. (1997) Thin Solid Films 305, 248-253).

It is well known in the art to sputter or evaporatively coat nanoscale structures such as microtubules with metals such as gold or platinum, respectively, in order to stabilize and image the structures. This, however, coats the entire field with metal, including the surface that supports the microtubule. In order to form conductive pathways, there is a need to metalize isolated microtubules or other nanoscale structures independent of their support.

Fritzche, W. et al. (Applied Physics Letters, 1999, 75, 2854-2856) describe the use of microtubules as a mask that protects a gold surface from being etched by a beam of argon ions. Following the dry etch, a band of gold metal remains where the microtubule was lying, whereas the remainder of the surface is gold free.

Metalization of bacterial rhapidosomes (Pazirandeh, M., et al., Biomimetics, 1, pg 41-50, 1992) and lipid-based tubule microstructures (Schnur, J. M. et al., Thin Solid Films, 152, pg 181-206, 1987; Markowitz, M. et al., Thin Solid Films, pg 242-7, 1993) was performed using a Pd-Sn colloidal solution or Pd(NH₃)₄Cl₂ respectively, to first form clusters of catalytic sites on the surface. The tubules were then coated with Ni, Co or Cu to form an electroless metal plating bath. Braun et al. (Braun, E. et al. (1998) Nature 391, 775-8) describe the metalization of DNA in which Ag+ ions were bound to the polyanionic polynucleotide stretched between two electrodes followed by reduction with hydroquinone to produce a conductive silver wire. WO 97/48837 and Kirsch, R., et al. (Thin Solid Films, 1997, 305, 248-253) describe the metalization of microtubules with Ni or Co using a Pd catalyst, with the need to establish a 10-15 nm layer to produce a continuum of metal. Ni-coated microtubules were shown to be conductive following electron-beam lithography connecting the microtubules to prestructured gold electrodes (Fritzche et al. (1999) Nanotechnology 10, 331-335.

Applicants have solved the deficiencies in the prior art by preparing a conductive, metalized nanoscale protein polymer without the need for a preliminary catalytic step using an additional metal.

### SUMMARY OF THE INVENTION

The invention provides an isolated metalized protein polymer wherein the polymer is coated with a single metal species. Preferred protein polymers are cytoskeletal microtubules prepared from tubulin and a preferred metal is gold.

In another embodiment the invention provides a process for the synthesis of a metalized protein polymer comprising:

a) providing a population of microtubules;

b) fixing the microtubules of (a) with a fixative whereby the fixative binds to the microtubules;

c) removing unbound fixative;

d) reacting the fixed microtubules of (c) with at least one reducible metal salt in the presence of a reducing agent wherein the microtubules are coated with the reducible metal of the metal salt to form a metalized microtubule.

In another embodiment the invention provides a conductive interconnect and a metal nano-wire comprising the metalized protein polymer of the invention.

### BRIEF DESCRIPTION OF THE FIGURES

FIG. 1a is a TEM image of glutaraldehyde-fixed, cysteine-quenched microtubules, stained with uranyl acetate

FIG. 1b is a TEM image of glutaraldehyde-fixed and dialyzed microtubules, stained with uranyl acetate

FIG. 2a is a TEM image of cysteine-quenched microtubules in suspension, gold coated in HAuCl₄ plus NaBH₄

FIG. 2b is a TEM image of cysteine-quenched microtubules in suspension, gold-coated in HAuCl₄ plus sodium ascorbate

FIG. 2c is a TEM image of cysteine-quenched microtubules in HAuCl₄ plus Na dithionite.

FIG. 3a is an SEM image of cysteine-quenched microtubules gold-coated in suspension and centrifuged onto a gap between bridging a central electrode and the inner of two concentric electrodes.

FIG. 3b is a graph illustrating the current-voltage measurement between the central electrode and the inner of the two concentric electrodes and between the inner and outer concentric electrodes.

FIG. 4a is a SEM image of glutaraldehyde-fixed and cysteine-quenched microtubules, gold-particle coated using HAuCl₄ and NaBH₄ while lying on a nickel TEM grid.

FIG. 4b is a SEM image of glutaraldeyde fixed and dialyzed microtubules, gold particle coated using HAuCl₄ and NaBH₄ while lying on the cross bar of a nickel TEM gird.

FIG. 5a is a SEM image of gold coated cysteine-quenched microtubules sintered at 400° C. under a N₂ atmosphere for one hour.

FIG. 5b is a TEM image of cysteine-quenched microtubules treated on a nickel TEM grid with GoldEnhance solution for 8 min following gold coating with HAuCl₄ plus NaBH₄.

FIG. 6a is a SEM image of cold-coated microtubules on nickel electrode of the same patterned chip as in FIGS. 6b and c.

FIG. 6b is a SEM image of the microtubules of FIG. 6a on the same nickel-patterned chip bridging two nickel electrodes across a 0.5 μm gap. After gold-enhancement with GoldEnhance.

FIG. 6c is a SEM image of the microtubules of FIG. 6b across the same 0.5 μm gap following annealing at 350° C. for one hour under an Ar atmosphere.

FIG. 7a is a graph of the current-voltage measurement of microtubules across the gap shown in FIGS. 6b and c before gold enhancement

FIG. 7b is a graph of the current-voltage measurement of the microtubules of FIG. 6b bridging the 0.5 μm gap shown in this Figure (following gold enhancement)

FIG. 7c is a graph of the current-voltage measurement of the microtubules of FIG. 6c bridging the 0.5 μm gap shown in this Figure (following annealing at 350° C. for one hour under an Ar atmosphere)

### DETAILED DESCRIPTION OF THE INVENTION

The invention relates to the metalization of protein polymers, principally for the production of nano-wires. Additionally, new methods for the metalization of protein polymers are disclosed.

The metalization of protein polymers is of use in the construction of nano-scale electronic devices, field emission displays, and conductive coatings.

The following definitions are used herein and should be referred to for claim interpretation.

As used herein the term “protein polymer” refers to a polymer of polypeptides comprised of amino acids. Typical protein polymers of the invention are microtubules.

The term “cytoskeleton microtubule” refers to a tubulin based protein polymer that is isolated from eukaryotic cells.

The term “metalized” refers to the coating of a substance or material with elemental metal. Within the context of the present invention protein polymers are “metalized” or coated either continuously or in an interrupted fashion with various elemental metals.

The term “single metal species” means a single metal element.

The term “aspect ratio” refers to the ratio of the length to the width of a protein polymer.

The term “fixative” refers to a substance that has the ability to crosslink the subunits of a protein polymer.

The term “conductive interconnect” refers to a material that establishes an electrically conductive bridge between two points.

The term “nano-wire” means a conductive interconnect with at least one dimension on the nanometer scale.

The term “gold enhancement” refers to the electroless deposition of gold onto a preexisting metal surface.

Metalized Biopolymers

The invention provides a metalized protein polymer that has been coated with a single metal species. Protein biopolymers of the invention include any protein or peptide that will adsorb a metal in a reductive fashion. A number of protein polymers are contemplated, including but not limited to structural proteins such as actin filaments, intermediate filaments, fibrin, collagen fibers, silk proteins, elastin, bacterial coat proteins and microtubules, as well as proteins that may function as a member of a binding pair such as avidin, streptavidin, immunoglobinbinding protein, immunoglobins, and receptor proteins.

Protein polymers that are particularly suitable are those that are rod-like or tubular in construction having a high aspect ratio and having a linear secondary structure. Aspect ratios of at least 10 are suitable where aspect ratios of up to 1000 are contemplated. Preferred protein polymers for use herein are those comprised of tubulin and will have length of about 10 microns in length or less, having an external diameter of about 4 nm to about 30 nm where about 25 nm is preferred and having an internal diameter of about 0 nm to about 20 nm, where about 14 nm is preferred.

The preferred protein biopolymer of the invention is a microtubule. Microtubules are cylindrical, hollow tubules of eukaryotic cells with diameters of about 25 nanometers and lengths in the micron range. They consist of protofilaments, built up by longitudinally associated α- and β-tubulin heterodimeric subunits. The protofilaments associate side to side to form the microtubule. Tubulin can be isolated from many eukaryotic cells. Nervous tissue, such as that found in mammalian brain, is a particularly rich source of tubulin. Tubulin from bovine brain tissue is plentiful, is easily purified and can be readily polymerized to form microtubules in vitro. Microtubules can easily depolymerize, however, so various capping and stabilization agents are needed to keep them intact. Methods for the isolation, purification and preparation of tubulin are described by Mitchison et al. (see GENERAL METHODS), Shelanski, M. L., et al. (Proc. Nat. Acad. Sci. USA, 70, 3, 1973, 765-768) and Murphy, D. B. (J. Biol. Chem., 261, 6, 1986, 2319-2324).

Protein polymers of the invention may be metalized with a variety of metals including noble or heavy metals gold, platinum, palladium and silver. The metalized protein polymer of the invention is typically coated with a single species of elemental metal, however it will be appreciated by the skilled person that combinations of metals are also possible. Depending on the method of coating, the protein may be continuously coated in metal, or may be coated in an interrupted fashion, where continuous coating is preferred. Preferred metalized protein polymers of the invention are microtubules comprised of tubulin having a continuous coating of gold.

Synthesis of Metalized Protein Polymers

Applicants provide a new method for the synthesis of a metalized protein polymer. In a typical reaction, microtubules are prepared from isolated tubulin by methods well known in the art (Mitchison, T. et al. as described in the GENERAL METHODS). Prepared microtubules are then contacted with a fixative which functions to crosslink the microtubules, increasing their stability to depolymerization and enhancing the reactivity of the surface for metal deposition. Suitable fixatives include but are not limited to di-aldehydes such as glutaraldehyde, keto-aldehydes and di-ketones, where glutaraldehyde is preferred. It is noted here that glutaraldehyde with only one end bound possesses a free reactive aldehyde group, which could allow it to function as a mild reducing agent, enhancing the reductive deposition of metal on the surface of the protein polymer. It is therefore contemplated that other fixatives having crosslinking functionality as well as having free reactive aldehyde groups will also be preferred fixatives. Typically, excess unreacted fixative is removed after reacting with the microtubules which may be accomplished by common methods such as dilution, quenching, washing, dialysis, centrifugation, and separation.

Metal for the bulk metalization of the protein polymer is provided in the form of a reducible metal salt. The metal salt is contacted with the fixed protein polymer in the presence of a suitable reducing agent and elemental metal is deposited on the surface of the protein. Suitable reducible metal salts include any of the salts of the metals of the noble metals including but not limited to gold, silver, platinum and palladium, where the salts HAuCl₄, AgNO₃, HPtCl₆, CuNO₃ and K₂PdCl₄ are preferred. Reducing agents useful for the reduction of the noble metals are well known in the art and a non-limiting list suitable in the present invention includes NaBH₄ and sodium ascorbate.

Typically the metalization reaction is terminated by any means known in the art (e. g. dilution, quenching, washing, dialysis, centrifugation, separation, and addition of an oxidant). Where it is desired to enhance the level of metalization on the protein polymer it is optionally possible to repeat the reductive metalization step to add more metal to the protein. Alternatively additional metal may be added by other gold enhancement methods. Gold enhancement protocols involve the electroless deposition of metal onto a preexisting metal surface. Materials and methods for gold enhancement are commercially available, see for example GoldEnhance EM solution (Cat no. 2113, Nanoprobes Inc., Yaphank, N.Y.).

The protein polymers of the invention may be metalized either in suspension or after being fixed or immobilized on a solid support. Immobilization offers the advantage of facile washing of the metalized protein following the metalization process and serves to produce metalized polymers that are much more linear and less entangled than those produced in suspension. Solid supports suitable for microtubule immobilization are well known in the art and include, nickel grids or disks, silicon wafers, carbon supports, aminosilane-treated silica and polylysine coated glass

Alternatively, polymers that have been metalized may be immobilized on a support in a patterned fashion for the construction of conductive interconnects and the like. Suitable supports for the immobilization of the metalized polymer include, but are not limited to nickel grids or disks, silicon wafers, carbon supports, synthetic polymer supports, substituted polystyrene, beads, agarose, nitrocellulose, and nylon as well as aminosilane-treated silica and polylysine coated glass. In a preferred embodiment, the microtubules are bound to a nickel surface such as Ni disks, TEM grids and electrodes.

Additionally, the metalized protein polymers may be further modified for use in nano-circuits and other nano-devices. For example the metalized protein may be heated to high temperature (sintered) to remove the protein scaffold and to more completely fuse the deposited metal into a stronger and more continuous wire. Another example is the use of gold or silver enhancing solution such as that supplied by Nanoprobes Inc. [Yaphank, N.Y.] (GoldEnhance EM and HQ Silver and LI Silver).

Conductive Interconnects

The gold-coated microtubules of the invention have been shown to have metallic (ohmic) behavior and may be adapted to show non-linear I-V characteristics. These behaviors lend themselves to the use of the metalized microtubules both as interconnects and as actual electronic devices (e,g, switches, logic gates). In the metallic case, the microtubules are expected to be able to link nanometer scale electronic devices together permitting the fabrication of high-density electronic circuits. It is contemplated that it will be possible to array the metallic microtubules in a crossed arrangement, where the distance between adjacent microtubules can be controlled by the potential difference between them, then the array could be used as a non-volatile memory device similar to that proposed by Leiber and collaborators (Rueckes T. et al. (2000). Science 289, 94-97) for carbon nanotubes. Non-linear conducting microtubules could find use in 3-terminal gated devices which can be used directly as switches, amplifiers or logic gates. By linking the gold particles of the metalized microtubules with organic semiconductors, it may be possible to develop 2-terminal switching devices, showing, for example, negative differential resistance (e.g. Fan et al. (2002) JACS 124, 5550-5560). Other possible applications include point sources for emission in field-emission display devices and as conductive inclusions in conductive coatings.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### General Methods

Unless otherwise specified, all the reagents were purchased from Aldrich Chemicals (Milwaukee, Wis.) and used without further purification. The meaning of abbreviations is as follows: “h” means hour(s), “min” means minute(s), “sec” means second(s), “d” means day(s), “mL” means milliliters, “L” means liters. “PIPES”: Piperazine-N,N′-bis[2-ethanesulfonic acid]. “EGTA”: Ethylene glycol-bis(β-aminoethyl ether)-N,N,N′N′-tetracetic acid. “GTP”: guanosine triphosphate. “PC”: phosphocellulose. “IB”: Injection Buffer

Recycled tubulin preparation and the large scale tubulin preparation were performed according to the methods and protocols of T. Mitchison, Harvard University, described in detail below.

Recycling Tubulin

Recycled tubulin fractions were stored at −80° C. after the phosphocellulose (PC) column and stored in small aliquots for day-to-day use. Recycled tubulin is generally stored in Injection Buffer (IB) without free GTP.

I. Solutions & Supplies

5X BRB80: (1X=80 mM K-Pipes, 1 mM MgCl2, 1 mM EGTA, pH 6.8 with KOH; store 5X BRB80 stock at 4° C.)

10X IB: 500 mM K-glutamate; 5 mM MgCl2, (pH of 1X˜7.0; store 10X stock at −20° C.; Cushion: 60% glycerol in 1X BRB80 (warm=37° C.)

100 mM GTP

Glycerol

Small Dounce (2 ml)

50.2Ti rotor (warm=37° C.)

TLA100.2 or 100.3 rotor (cold=2-4° C.)

II. Recycling Protocol

1. Thaw 3-4 3 ml PC column fractions at 37° C. Transfer to ice and mix in a 50 ml conical.

2. Add BRB80 to 0.5X, 4 mM MgCl2 and 1 mM GTP where the tubulin aliquots are in CB (50 mM K-Pipes, pH 6.8; 1 mM EGTA; 0.2 mM MgCl2). Thus, the final buffer composition is 90 mM K-Pipes, pH 6.7, 1.5 mM EGTA, 4.2 mM MgCl2, 1 mM GTP. Mix by swirling and store on ice for 5′.

3. Transfer to 37° C. After 2′ add half volume of glycerol. Mix the glycerol in by gentle vortexing. Incubate at 37° C. for 40′.

4. Layer on a warm cushion (10-14 ml) in two 50.2Ti (or equivalent) tubes. Spin for 45′ at 40K at 35° C. in a 50.2 rotor.

5. Aspirate sample, rinse sample cushion interface with warm IX IB 2-3 times. Aspirate completely and rinse the tubulin pellet 2X with warm 1X IB to remove any residual glycerol. Do this while the pellet is held in a 37° C. bath—fill tube about half-way with warm IB (rinsing sides and pellet well), then aspirate and repeat.

6. Resuspend pellet in 1-2 ml of ice cold IB (the exact volume will depend on the concentration and polymerizability of the tubulin in the column fractions). Transfer the chunky pellet in IB to an ice-cold 2 ml dounce. Gently dounce on ice to break up the chunks. Incubate on ice for 30′. During this cold depolymerization, keep douncing gently every 2′-3′. Gentle sonication can also be used to break up the pellets.

7. Spin 90K in TLA100.2 or TLA100.3 rotor at 2° C. for 10′-15′ to clarify the depolymerization mix.

8. Collect supernatant on ice and measure A280 of a 1/100 dilution in IB. Calculate concentration of tubulin using an extinction coefficient at 280 nm of 115,000 M-1 cm-1. Freeze in 10-50 μl aliquots in liquid nitrogen and store at 80° C. (Note: 1 mg/ml tubulin=10 μM)

Large Scale Tubulin Preparation

Tubulin is purified from bovine/porcine brain by two cycles of polymerization/depolymerization followed by removal of copurifying proteins on a phosphocellulose (PC) column. The procedure described here is for a large scale prep (10 cow brains) that yields 1-4 grams of tubulin.

I. Tubulin Prep Outline

1. Remove meninges, brain stems and blood clots, weigh and homogenize brains in blenders

2. Clarify homogenate and use supernatant for 1st polymerization cycle

3. Collect 1st cycle polymer fraction by centrifugation

4. Depolymerize 1st cycle polymer by homogenization at 0-4° C.

5. Clarify depolymerization mix and use supernatant for 2nd polymerization cycle

6. Collect 2nd cycle polymer fraction by centrifugation

7. Depolymerize 2nd cycle polymer by homogenization at 0-4° C.

14. Clarify depolymerization mix and load supernatant onto PC column

15. Collect flow through from PC column, aliquot and freeze at −80° C.

II. Buffers & Nucleotides

PB (Pipes/Polymerization Buffer): 0.1 M K-Pipes, pH 6.8, 0.5 mM MgCl2, 2 mM EGTA, 0.1 mM EDTA, 0.1% b-mercaptoethanol, 1 mM ATP. Need 8 liters in cold-room(Add ATP and BME just prior to beginning the prep) CB (Column Buffer): 50 mM K-Pipes, pH 6.8; 1 mM EGTA; 0.2 mM MgCl2. Need ˜25 liters for equilibration, running and storage of PC column CB+1 M KCl: Need ˜10 liters for pre-washing and eluting the PC column

To make 1 L of 10X CB:

151.2 grams PIPES, free acid

3.8 grams EGTA

2 ml of 1 M MgCl2

pH with KOH to pH 6.75, and bring up to 1 liter.

Check pH at 1X is 6.7 Make 3.5 liters of 10X CB for 10-12 brain prep.

GTP: Sigma Type IIS-#G-8752

ATP: Sigma Grade 1-#A-2383

Glycerol: 2-3 L prewarmed to 37° C. (store overnight in 37° C. incubator)

III. Pouring a 1 L Phosphocellulose (PC) Column

Resin: Whatman P11 Cellulose Phosphate—fibrous cation exchanger (1 gram of PC swells to about 4 ml packed resin)

Summary: To pour a 1 L column, start with 220 grams dry resin divided into 5 aliquots of 44 grams. Treat each aliquot with acid/base in a 2 L beaker as described below. Gently stir the resin with a plastic/glass rod to suspend it in a 2 L beaker and then allow the resing to set for 5 min. by gravity. De-fine of the resin using a standard acid/base cycling protocol. After acid/base treatment the resin is washed well, packed, treated with BSA to block irreversible binding sites and equilibrated for use.

Solutions & Supplies:

220 grams Phosphocellulose

5 L 0.5 N NaOH

5 L 0.5 N HCl

13 L 0.5 M K-Phosphate, pH 6.8

5 L ddH20

5 2 L beakers

12 L CB+1M KCl

20 L CB

300 ml of 30 mg/ml BSA in CB (filtered)

1-1.5 L cleaned column housing

2 stirring rods

2 aspirators with large traps

Peristaltic pump

10 ml plastic pipets as inlets for peristaltic pump

Column Preparation Procedure:

1. Pour 1 L 0.5 N NaOH into 5×2 L beakers. Add 44 grams PC to each beaker stirring gently with a rod until the PC is wetted and an even slurry is present. Let stand at room temperature for 5 min

2. Aspirate off supernatant, including fines, and quickly add 1 L 0.5M K-phosphate to neutralize, gently mixing with a rod. Check that pH is ˜7 and let stand 5 min.

3. Aspirate off supernatant, add 1 L ddH20 and gently stir to resuspend settled resin.

4. Allow the resin to settle.

5. Aspirate off supernatant, add 1 L 0.5N HCl, gently stir to resuspend and wait 5 min.

6. Aspirate off supernatant, add 1 L 0.5M K-phosphate, stir and check pH is 7.

7. After resin has settled, aspirate supernatant and combine all the resin in a 4 L beaker. Use the remaining 0.5M K-phosphate to wash the resin by resuspending, letting settle and aspirating the supernatant.

8. Wash 3×1 L CB+1M KCl as done in 7.

9. In the cold room, pour the resuspended resin into the column housing (with a mark approx. 50 cm high in a 5 cm ID housing) and pack by pumping from the bottom(i.e. the peristaltic pump is “sucking” buffer from the bottom of the column and depositing it into a waste jug). Pack at 45 ml/hour/cm cross-sectional area. For a 5 cm diameter column this is ˜880 ml/hour or ˜14.5 ml/min. After resin is packed, switch to pumping from the top. Run 7 L of CB+1M KCl through the column at 5-10 ml/min.

10. Wash with 10 L CB. Check conductivity to ensure that all the KCl is gone. The resin may expand as the salt is washed out so make sure there is a large buffer head on the resin bed

11. Load 300 ml of 30 mg/ml BSA (Fraction V; filtered) in CB, follow with 700 ml CB and stop the column. Leave the column sitting for 2 hr during which the BSA blocks irreversible binding sites on the resin—this is very important the first time a column is used to prevent loss of the tubulin.

12. Wash the column with 2 L CB+1M KCl to elute BSA that is not irreversibly bound.

13. Wash column with 10 L of CB. The column is now ready for use.

IV. Brains

It is essential to get fresh brains. Frozen brains do not work for preparing tubulin. It is preferred if the brains are freshly removed and transported in an ice-filled cooler to the lab within 1-2 hours of removal.

V. Centrifuges & Rotors

6 Sorvall RC-5C or equivalent low-speed centrifuges

6 GSA or equivalent rotors (cold)

4 Beckman ultracentrifuges

4 Type 19 rotors (warm)

2 Type 35 rotors (warm)

2 Type 45Ti rotors (cold; need to warm up one after 1st cold use)

1 Type 50.2Ti rotor (cold)

cold=4° C. (put overnight in cold room)

warm=37° C. (put the rotors overnight in a large bacterial shaker set to 37° C.)

VI. Protocol

1. In the cold room, remove meninges (membrane surrounding the brain; best done by using paper towels to “blot” the brain surface), blood clots, and brain stems; weigh the brains and homogenize with equal volume of PB for 3×15 s in a Waring blender.

2. Collect homogenate (˜8-9 liters), transfer into 36 GSA bottles and spin 90′ at 12K in a GSA rotor at 4° C.

3. Collect supernatant and transfer 1 liter to a 1.8 L glass Fernbach flask that has 500 ml of 37° C. glycerol. Add 0.1 mM GTP, 0.5 mM ATP, and 3.5 mM MgCl2 (this gives 0.1 mM GTP, 1.5 mM ATP and 4 mM MgCl2 final). The ATP and GTP are added as solids. Hold the flask in a warm water-filled sink and swirl constantly to dissolve the solids and to mix in the glycerol. Transfer to a 37° C. bath, monitor temperature of mixture using a clean thermometer and polymerize for 60 min after the temperature of the sample has reached 32° C. The approach to 37° C. can be accelerated by swirling the flask in a large hot water (˜50° C. temperature) reservoir—constant swirling is essential in this case to disperse the heat evenly and care must be taken to avoid overheating the mixture.

4. Transfer the polymerization mixture to Type 19 bottles, and spin for 2.5 hrs at 19K in 4 Type 19 rotors at 35° C. Use an additional Type 35 at 17.5K for 2.5 hrs if necessary. At the end of the spin set centrifuges to 4° C.

5. Decant and discard supernatant. In the cold room, resuspend the gelatinous pellets in PB aiming for a final volume of ˜700-800 ml. Use ˜40-50 ml for 3 tubes, sequentially removing the pellets from each tube using a plastic scraper and making sure that all tubes get rinsed once or twice after the majority of the large gelatinous pellet has been removed. To homogenize the chunky pellet resuspension, use a Yamato “pour-through” continuous flow homogenizer—this is a device that drives a motorized teflon pestle in a funnel shaped glass barrel. Mixtures poured on top get homogenized by the pestle as they travel through the middle of the barrel and come out the bottom. “Yamato” the chunky pellets till the resuspension is a smooth yellow liquid of ˜700-800 ml total volume. After all the pellets are homogenized, depolymerize on ice for ˜30 min during which “Yamatoing” is continued once every 3-5 min. Alternatively a large motorized teflon dounce or large tip sonicator can be used as alternatives to the Yamato for resuspending the chunky pellets. Check protein concentration by Bradford using BSA as a standard. If >20 mg/ml, dilute to 20 mg/ml.

6. Spin the depolymerization mixture 30 min at 35K in 2 Type 45 rotors at 4° C. At the end of the spin set the centrifuges to 35° C.

7. Decant supernatant into a 1 L graduated cylinder in cold-room and measure volume. Pour into a 1.8 L Fernbach flask, add half volume of 37° C. glycerol, solid GTP to 0.5 mM final and MgCl2 to 4 mM final (additional 3.5 mM). Set up polymerization as described in 3. above. Polymerize for 40 min after temperature of mixture has reached 32° C.

8. Spin the polymerization mixture at 35K at 35° C. for 1 hr in 2 Type 35s+1 Type 45.

9. Discard supernatant and resuspend pellets in a final volume of ˜100-150 ml of CB, as described above in 5. Protein concentration by Bradford should not be more than 25 mg/ml. Resuspend pellets in a final volume of ˜100-150 ml. Depolymerize on ice for 40 min and then spin the depolymerization mix30′ at 40K in a 50.2Ti rotor at 4° C.

10. Collect supernatant, measure concentration by Bradford and load onto the PC column (approx. 50 cm high X 5 cm ID=˜1 L; Flow rate=6 ml/min). After the sample is loaded and ˜150 ml buffer has flowed through start collecting fractions. The eluted tubulin will be apparent by its slight yellowish tinge. Measure concentration by Bradford using BSA as a standard and pool such that the final concentration is between 5-10 mg/ml. Mix pool on ice, make 3 ml aliquots in 5 ml snap-cap polypropylene tubes and freeze in liquid nitrogen. Store frozen aliquots at −80° C. The entire procedure, from time of arrival of brains till freezing of the tubulin will take ˜17-18 hours. The next day, run 3 volumes of CB+1M KCl to elute MAPs from the PC column (these can be collected if desired), and then equilibrate column into CB+0.1% azide for storage. Phosphocellulose will lose capacity when stored wet—this can be reduced by storage in a phosphate buffer (50 mM phosphate, pH 7 with 1 mM EGTA and 0.2 mM MgCl2) containing 0.1% azide.

### EXAMPLE 1

### Preparation of Microtubules

Recycled bovine tubulin was prepared according to the protocols as described in the GENERAL METHODS and stored at a concentration of 10 mg/ml in BRB80 buffer (80 mM K⁺-PIPES (Piperazine-N,N′-bis[2-ethanesulfonic acid])+1 mM MgCl₂+1 mM EGTA (Ethylene glycol-bis(β-aminoethyl ether)-N,N,N′N′-tetracetic acid).

Briefly, the preparation of recycled tubulin involves the homogenization of bovine brain and subjection of the homogenized material to a succession of differential centrifugations and polymerizations and depolymerizations and passage through a phosphocellulose column where it is purified free of contaminating proteins. The preparation of tubulin (total 0.42 g) was aliquotted out in 2-ml fractions (6.35 mg/ml) and stored frozen. Eight ml were then subjected to two additional rounds of additional polymerization and depolymerization and were aliquotted out in 10 and 50 ul fractions and frozen, to be thawed individually for experiments. SDS-PAGE showed the preparation to be homogeneous in α- and β-tubulin. The recovery of tubulin following recycling was ˜25%.

The recycled tubulin was dissolved in BRB80 plus 22% glycerol containing 4 mM MgCl₂ and 1 mM GTP (guanosine triphosphate) at 0° C. and left for 5 min., after which polymerization was induced by incubation at 37° C. for 15 min. The microtubule suspension was then made 50 μM in paclitaxel (Sigma, St. Louis, Mo.) and left for 5 min at room temperature. At this point, the microtubule suspension was diluted 1:10 with 1.1% glutaraldehyde (Electron Microsopy Supplies, West Chester, Pa.) plus 10 μM paclitaxel in BRB80 and left for 4 min at room temperature to fix the microtubules. The suspension was then diluted 25-fold with BRB80 containing 10 μM paclitaxel or with BRB80 containing 50 mM cysteine (or 50 mM glycine) and 10 μM paclitaxel. This suspension was left at room temperature for several hours.

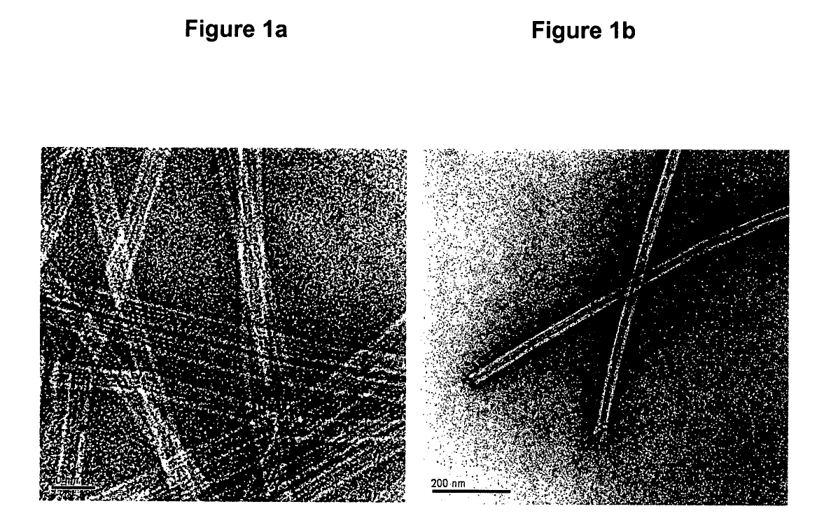

Lucite® inserts on which rested 12 mm cover slips were placed in centrifuge tubes of an SW40 swinging bucket rotor (Beckman). BRB80+10% glycerol+10 μM paclitaxel (1.5 ml) was added and overlaid with 6.5 ml of the microtubule suspension. The tubes were then centrifuged in a SW40 swinging bucket rotor for 1 h at 20,000 rpm at 25° C. at which point the upper layer was aspirated and the interface washed two times with BRB80. The remaining liquid was aspirated to within 2 mm of the coverslip at which point the tube was swirled to gently resuspend the microtubules. The microtubules were either used directly or dialyzed 1:100 against BRB80 overnight at room temperature for those that were not quenched with cysteine or glycine. TEM images of uranyl acetate stained microtubules showed little difference between the different preparations (FIGS. 1a and 1b). For long term storage, both quenched and unquenched microtubules were aliquotted into 50 μl volumes and frozen in liquid N₂. Rapid freezing and thawing had little effect on the intactness of the microtubules as observed by TEM.

### EXAMPLE 2

### Metalization of Microtubules in Suspension—Cysteine-quenched Microtubules in Suspension Gold Coated in HAuCl₄ Plus NaBH₄

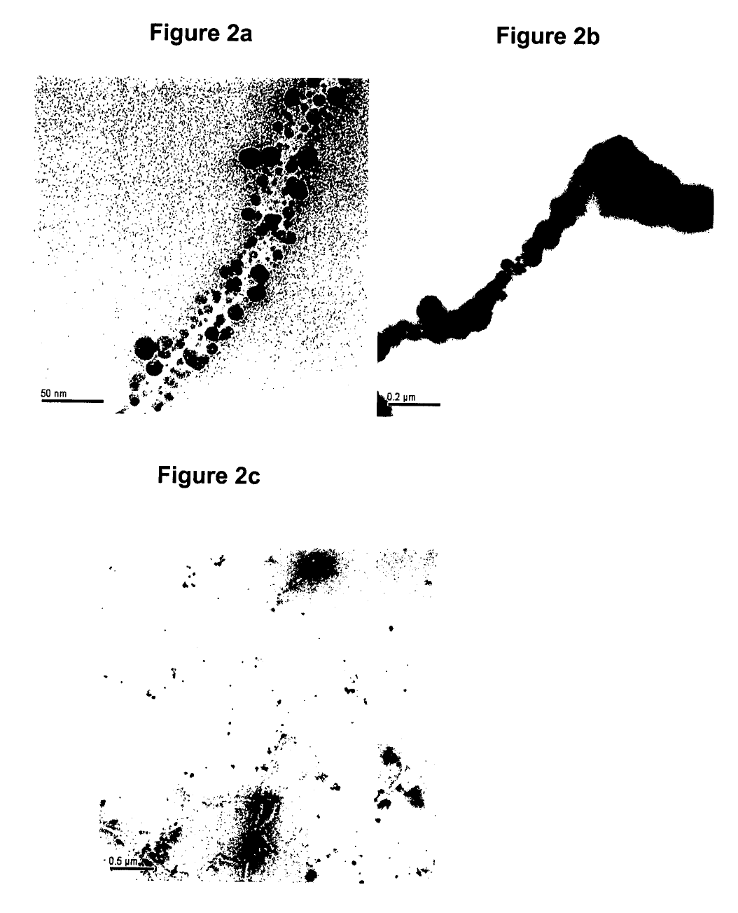

The microtubule suspension (50 μl of a 0.5 mg/ml fixed and cysteine quenched microtubules) was added to 5 ml of distilled water. Five ml HAuCl₄ (1 mM), and 5 ml NaBH₄ (2.7 mM) were added dropwise at equal rates over a period of 15-30 min to give a final volume of 15 ml. The color of the resulting suspension ranged from salmon red to purple. Several μl of the metalized microtubules were placed on a Ni TEM (Transmission Electron Microscopy) grid for examination by transmission electron microscopy (FIG. 2a).

### EXAMPLE 3

### Metalization of Microtubules in Suspension—Cysteine-quenched Microtubules in Suspension Gold Coated in HAuCl₄ Plus Sodium Ascorbate Acid

The microtubule suspension (50 μl of a 0.5 mg/ml fixed and cysteine quenched microtubules) was added to 5 ml of distilled water. Five ml HAuCl₄ (1 mM), and five ml sodium ascorbate (2.4 mM) were added dropwise at equal rates over a period of 15-30 min to give a final volume of 15 ml. The color of the resulting suspension ranged from salmon red to purple. Several μl of the metalized microtubules were placed on a Ni TEM grid for examination by transmission electron microscopy (FIG. 2b).

### EXAMPLE 4 (COMPARATIVE)

### Metalization of Microtubules in Suspension—Cysteine-quenched Microtubules Treated in HAuCl₄ Plus Na Dithionite

The microtubule suspension (50 μl of a 0.5 mg/ml fixed and cysteine quenched microtubules) was added to 5 ml of distilled water. Five ml HAuCl₄ (1 mM), and five ml Na dithionite (2.6 mM) were added dropwise at equal rates over a period of 15-30 min to give a final volume of 15 ml. The color of the resulting suspension ranged from salmon red to purple. Several μl of the metalized microtubules were placed on a Ni TEM grid for examination by transmission electron microscopy (FIG. 2c).

In the case of NaBH₄ reduction, TEM indicated the presence of gold particles <20 nm in diameter coating the outside of the microtubules, shown in FIG. 2a. The gold coverage was considerably thicker in the case of sodium ascorbate with gold particles appearing to fuse together, shown in FIG. 2b. In contrast, the gold coverage was sparse for Na dithionite with only rare adhesion of gold particles to the microtubule surface, shown in FIG. 2c. Some distortion of the microtubules from linearity was observed upon gold deposition in suspension.

### EXAMPLE 5

### Construction of Conductive Interconnect

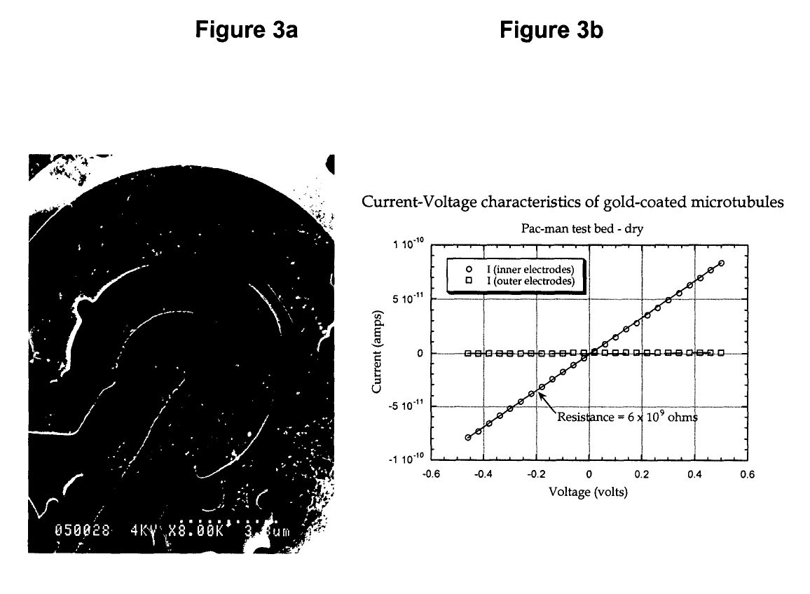

Microtubules, quenched using cysteine and gold-coated using HAuCl₄ plus NaBH₄, were spun down onto a gold-patterned silicon chip in a SW 40 rotor. The chip was placed face-up on top of a Lucite® insert located in the bottom of the centrifuge tube. Twenty μl of the metalized microtubule suspension was then placed in the centrifuge tube and the tube spun in an SW40 rotor for 1 h at 20,000 rpm. The metalized microtubules were somewhat inhomogeneously dispersed on the surface of the patterned chip, some of which bridged gaps between gold electrodes. This preparation was used for electrical measurements. An SEM (Scanning Electron Microscopy) image of the gold particle-coated microtubules bridging the gap between a central gold electrode and the inner of two concentric electrodes on silicon is shown in FIG. 3a. The gap between the inner and outer concentric electrodes is not bridged by the microtubules. The current-voltage measurements corresponding to the inner and outer gaps is shown in FIG. 3b. The inner gap is conductive, showing ohmic resistance in the gigaohm range while the outer gap is an open circuit, consistent with the SEM image.

### EXAMPLE 6

### Metalization of Microtubules on a Surface—Glutaraldehyde-fixed and Cysteine-quenched Microtubules.

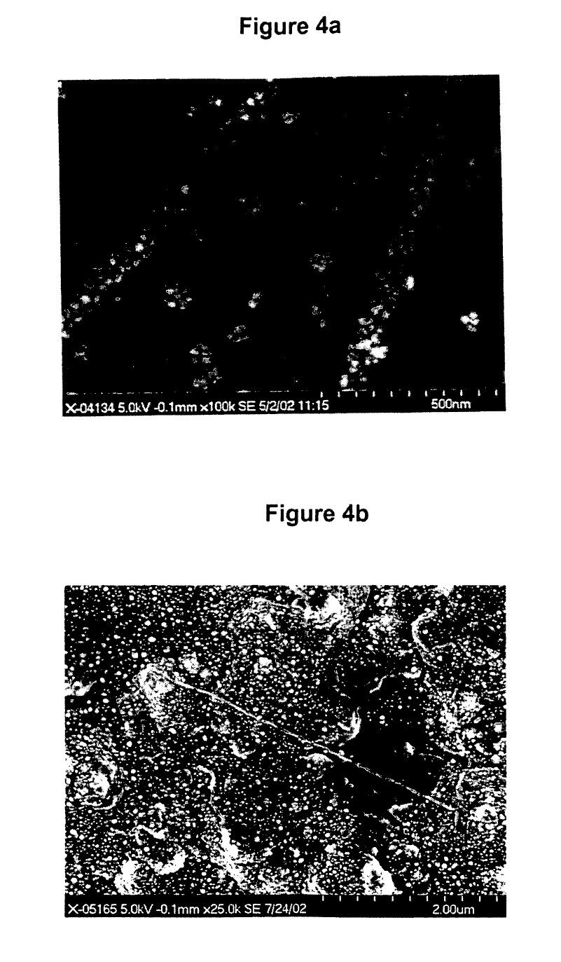

Microtubules were found to bind and metalize well on Ni surfaces, either Ni TEM grids or to Ni disks. A 5 μl drop of suspension (˜0.5 mg/ml glutaraldehyde-fixed, cysteine-quenched microtubules) was left for 10 min on the surface and then wicked off. Microtubules could be observed on the Ni grids by TEM (FIG. 1). The grids and disks were suspended using forceps in 5 ml of distilled water. Five ml HAuCl₄ (1 mM) and 5 ml NaBH₄ (2.7 mM) were added dropwise at equal rates over a period of 15-30 min to give a final volume of 15 ml. SEM observation of the grids (FIG. 4, top) and disks (not shown) revealed linear microtubules completely coated with gold particles of <20 nm.

A 5 μl drop of suspension (˜0.5 mg/ml glutaraldehyde-fixed and dialyzed microtubules) was left for 10 min on the surface and then wicked off. Microtubules could be observed on the Ni grids by TEM (FIG. 1). The grids and disks were suspended using forceps in 5 ml of distilled water. Five ml HAuCl₄ (1 mM) and five ml NaBH₄ (2.7 mM) were added dropwise at equal rates over a period of 15-30 min to give a final volume of 15 ml. SEM observation of the grids (FIG. 4b) and disks (not shown) revealed linear microtubules completely coated with gold particles of <20 nm.

There was a high degree of specific binding of the gold particles to the microtubules. Gold coating of the glutaraldehyde-fixed and dialyzed microtubules appeared to give a denser coating with gold particles on average of smaller size than was observed for the glutaraldehyde-fixed and cysteine-quenched microtubules. Gold metalization on the Ni surfaces (FIGS. 4a and b) gave more linear metalized microtubules than those in suspension (FIGS. 2 and 3), probably because of their immobilization by the surface. No derivativization of the Ni surface was necessary to potentiate microtubule adherence.

### EXAMPLE 7

### Further Processing Following Microtubule Metalization

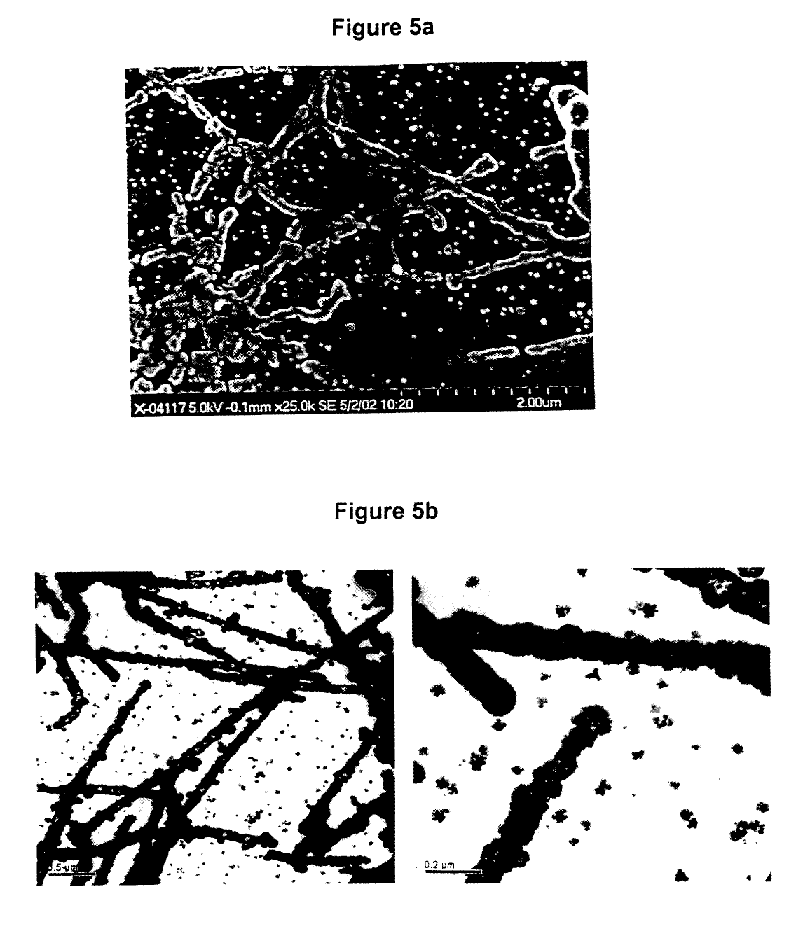

Gold particle-coated microtubules lying on a Ni surface could be sintered to produce a continuum of gold. Microtubules on the cross bars of a Ni TEM grid coated with <20 nm gold particles (FIG. 4) were heated for 1 h at 400° C. under a N₂ atmosphere. SEM (FIG. 5a) indicated the fusion of the gold particles to give a continuum of gold. Suspended microtubules formed wire-like structures with continuous gold coverage. Microtubules lying on the Ni surface showed those that had continuous gold coverage but also those that gave lengths of gold separated by occasional gaps. These reflect some competition between surface wetting and fusion of the gold particles with each other. This competition should in principle be controllable by changing the atmosphere in which the fusion occurs or in modifying the nature of the surface.

Gold particle-coated microtubule were also be treated with GoldEnhance EM solution (Cat no. 2113, Nanoprobes Inc., Yaphank, N.Y.) that gave further gold deposition onto the preexisting gold particles causing them to fuse to each other (FIG. 5b).

### EXAMPLE 8

### Electronic Properties of Microtubule Gold-coated on a Surface

Frozen glutaraldehyde-fixed non-quenched and dialyzed microtubules were thawed and diluted 4-fold with BRB-80. Ten microliters of this suspension of microtubules were placed on a nickel-patterned chip, previously cleaned by glow discharge. The droplet was left for ten minutes at room temperature on the chip in a petri plate containing air at high relative humidity. The chip was then wicked dry and the chip allowed to air dry at ambient relative humidity. The chip was then held by a self-locking forceps and suspended in a scintillation vial containing 5 ml of deionized water. HAuCl₄ (1 mM) and NaBH₄ (2.4 mM) were added dropwise at equivalent rates until 5 ml of each had been delivered over a 30 minute period to the vial without stirring. The chip was then dipped into deionized water and allowed to air dry.

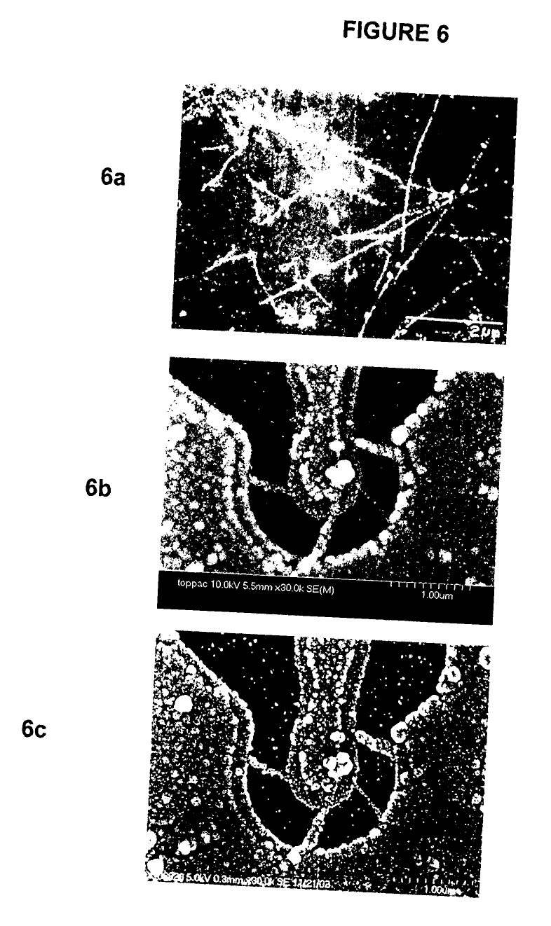

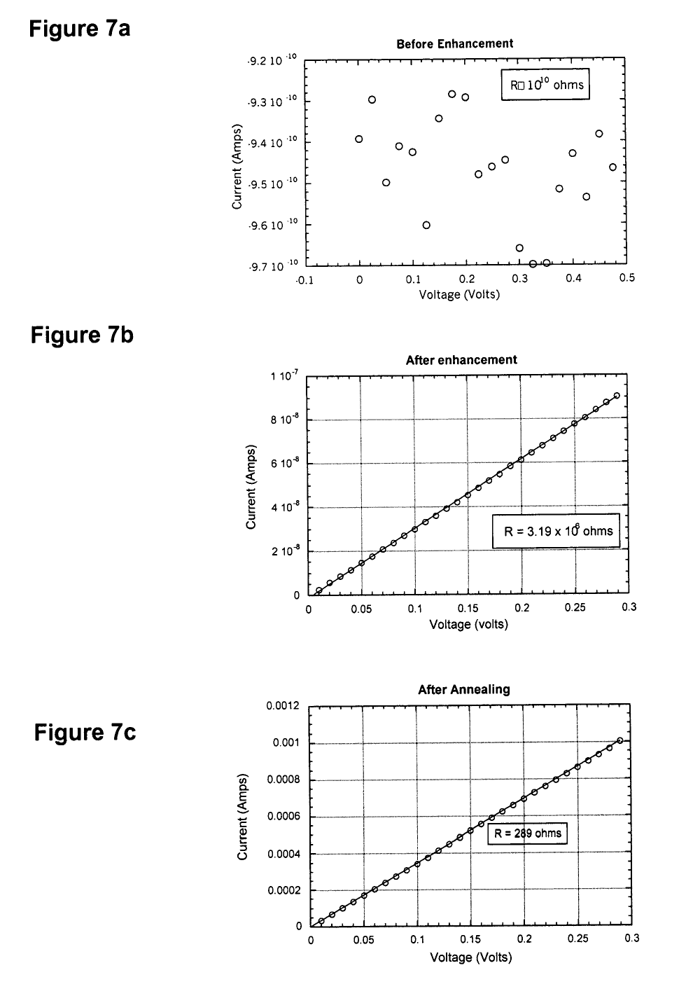

A current-voltage measurement (I-V) was conducted on the microtubules of FIG. 6a across a 0.5 μm gap between a pair of electrodes shown in FIG. 6b and c. The voltage sweep was from −0.5 V to +0.5 V (FIG. 7a) reveals a resistance of >10¹⁰ ohms.

Twenty microliters of GoldEnhance (Nanoprobes Inc.) was placed on the chip and left for 10 minutes, after which the chip was rinsed with water and wicked dry (FIG. 6b).

Another I-V measurement was conducted across the same gap as above (FIG. 7b) indicating a resistance of 3.2×10⁶ ohms. The chip was annealed at 350° C. under an Ar atmosphere for one-hour (FIG. 6c). Another I-V measurement was conducted across the same gap as above (FIG. 7c) indicating a resistance of 290 ohms.

What is claimed is:

1. An isolated metalized protein polymer wherein the polymer is coated with a single metal species.

2. The isolated metalized protein polymer of claim 1 wherein the protein polymer is selected from the group consisting of actin fibers, intermediate filaments, fibrin, collagen fibers, silk proteins, elastin, bacterial coat proteins, microtubules, avidin, streptavidin, immunoglobin binding protein, immunoglobins, and receptor proteins.

3. The isolated metalized protein polymer of claim 2 wherein the protein polymer has an aspect ratio greater than 10.

4. The isolated metalized protein polymer of claim 3 wherein the protein polymer is tubular in structure.

5. The isolated metalized protein polymer of claim 4 having a length of 10 microns or less, having an external diameter of about 4 nm to about 30 nm and having an internal diameter of about 0 nm to about 20 nm.

6. The isolated metalized protein polymer of claim 5 wherein the protein polymer comprises tubulin.

7. The isolated metalized protein polymer of claim 1 wherein the coating of the protein polymer is interrupted.

8. The isolated metalized protein polymer of claim 1 wherein the coating of the protein polymer is continuous.

9. The isolated metalized protein polymer of claim 1 wherein the single metal species is selected from the group consisting of gold, silver, platinum and palladium.

10. An isolated microtubule wherein the microtubule is continuously coated with gold.

11. A process for the synthesis of a metalized microtubule comprising:
a) providing a population of microtubules;
b) fixing the microtubules of (a) with a fixative whereby the fixative binds to the microtubules;
c) removing unbound fixative;
d) reacting the fixed microtubules of (c) with at least one reducible metal salt in the presence of a reducing agent wherein the microtubules are coated with the reducible metal of the metal salt to form a metalized microtubule.

12. A process according to claim 11 wherein the metalized microtubule is optionally isolated after step (d).

13. A process according to claim 11 wherein step (d) is repeated one or more times to enhance metal coating with the same or different metal.

14. A process according to claim 11 wherein the removal of unbound fixative is accomplished by a method selected from the group consisting of dilution, washing, dialysis, centrifugation, and separation.

15. A process according to claim 11 wherein the population of microtubules are immobilized on a solid support.

16. A process according to claim 11 wherein the population of microtubules are in suspension.

17. A process according to claim 11 wherein the solid support is selected from the group consisting of a nickel grids, nickel disks, silicon wafers, carbon supports, aminosilane-treated silica and polylysine coated glass.

18. A process according to claim 11 wherein the metalized microtubule of step (d) is optionally immobilized on a solid support.

19. A process according to claim 18 wherein the solid support comprises a material selected from the group consisting of nickel, silicon, carbon, substituted polystyrene, agarose, nitrocellulose, nylon, aminosilane-treated silica and polylysine coated glass.

20. A process according to claim 11 wherein the fixative acts to cross-link the microtubules.

21. A process according to claim 11 wherein the fixative is selected from the group consisting of di-aldehydes, keto-aldehydes and di-ketones.

22. A process according to claim 11 wherein the unbound fixative is removed by a process selected from the group consisting of dilution, washing, dialysis, centrifugation, separation, and quenching.

23. A process according to claim 11 wherein the reducible metal salt is selected from the group consisting of salts of the metals selected from the group consisting of gold, silver, platinum and palladium.

24. A process according to claim 23 wherein the reducible metal salt is selected from the group consisting of HAuCl₄, AgNO₃ H₂PtCl₆, and K₂PdCl₄.

25. A process according to claim 11 wherein the reducing agent is selected from the group consisting of NaBH₄, and sodium ascorbate.

26. A process according to claim 11 wherein the reaction of (d) is optionally terminated before the reaction runs to completion.

27. A conductive interconnect comprising the metalized protein polymer of claim 1.

28. A metal nano-wire produced by a process comprising sintering the metalized protein polymer of claim 1.

29. A method of producing a nano-wire comprising sintering the metalized protein of claim 1.

30. A metal nano-wire produced by a process comprising gold enhancing the metalized protein polymer of claim 1.

31. A process of producing a nano-wire comprising gold enhancing the metalized protein of claim 1.
